(19) United States
(12) Patent Application Publication
(10) Pub. No.: US 2009/0196828 A1
(43) Pub. Date: Aug. 6, 2009

(54) CONTRAST AGENT COMPRISING A TM2+ CONTAINING LUMINESCENT SUBSTANCE FOR OPTICAL IMAGING

(75) Inventor: Jan Frederik Suijver, Dommelen (NL)

Correspondence Address: PHILIPS INTELLECTUAL PROPERTY & STANDARDS, P.O. BOX 3001, BRIARCLIFF MANOR, NY, 10510, US

(73) Assignee: KONINKLIJKE PHILIPS ELECTRONICS N.V., EINDHOVEN (NL)

(21) Appl. No.: 12/438,566

(22) PCT Filed: Aug. 15, 2007
(86) PCT No.: PCT/IB2007/053245

§ 371 Date: Feb. 24, 2009

(30) Foreign Application Priority Data
Aug. 25, 2006 (EP) 06119521.0

(51) Int. Cl. A61K 49/00; C12Q 1/02; C01F 17/00; A61B 6/00

(52) U.S. Cl. 424/9.6; 424/9.1; 435/29; 423/263; 600/431

(57) ABSTRACT

The invention relates to a contrast agent for optical imaging. The inventive contrast agent comprises a luminescent substance, wherein the luminescent substance comprises Tm²⁺. Furthermore, the present invention refers to the use of a Tm²⁺ containing material as a luminescent substance in a contrast agent for optical imaging. The invention also relates to a method of optical imaging of tissue, the method comprises the steps (a) contacting an effective amount of the Tm²⁺ containing contrast agent with the tissue, (b) exposing the tissue to electromagnetic radiation in the wave-length range between 200 nm and 800 nm, (c) detecting any luminescence signal emitted by the tissue exposed to the electromagnetic radiation, and (d) processing the detected luminescence signal(s) into an image.

The present invention relates to the field of optical imaging. The invention provides new contrast agents for imaging cells, tissues and organs in vivo and in vitro. In particular, contrast agents containing luminescent substances are provided to improve the imaging of tissue by optical imaging techniques.

Medical imaging techniques have become more and more important over the last few years. Important imaging techniques include, for example, Positron Emission Tomography (PET), Single Photon Emission Computed Tomography (SPECT), Magnetic Resonance Imaging (MRI) and Computed Tomography (CT). All these techniques are characterized by a significant complexity with respect to the employed equipment. Besides the huge purchase costs of the corresponding apparatuses, the high running costs negatively influence the expenses per examination of patient. Moreover, the analyses or evaluation of obtained imaging data is complex and can only be performed by experts.

In contrast, optical imaging techniques involve significantly less technical and personal effort compared to the aforementioned techniques. Therefore, optical imaging techniques for e.g. the assessment of tissue anatomy or for imaging organs or metabolic and molecular functions are of major interest and huge efforts have been made to improve the quality of optical imaging.

The term “optical imaging” encompasses imaging techniques where light, preferably a light source emitting in the ultraviolet (UV) spectral region, the visible (VIS) spectral region and/or the near-infrared (NIR) spectral region is used for characterizing or imaging tissues, cells or organs. The interaction of photons with e.g. tissue is based on the absorption of the light, the scattering of light and the emission of luminescence. As in the established imaging methods, such as CT, MRI or ultrasonic imaging, exogenously applied optical contrast agents principally provide the opportunity of generating disease specific signals within the tissue, thus enabling the display of physiological and molecular conditions which are characteristic for a certain diseased state and progression. In principle, contrast enhancement is achieved when an administered agent changes the imaging properties of the diseased tissue to a different extent relative to the surrounding normal tissue. Consequently, a “contrast agent” may be understood as an agent, which changes the absorption, scattering or the luminescent properties of the diseased tissue to a different extent relative to the surrounding normal tissue.

A fundamental observation for optical imaging procedures relates to the fact that the absorption of light in tissue, e.g. absorption originating from oxy- and deoxy-haemoglobin or other porphyrins leads to tissue autofluorescence throughout the visible (VIS) spectral region up to approximately 700 nm. However, most of the available contrast agents or luminescent agents emit in the aforementioned visible spectral range and especially in the blue or green spectral range. It is obvious that direct in vivo applications may be compromised if blood and human tissue provide broadband emissions in the same spectral range. The resulting overlap of the autofluorescence and the emission from the luminescent contrast agent contained in the tissue may significantly affect the quality of the resulting imaging.

Given the relatively low absorbance and scatter of living tissue in the near infrared region of the spectrum, considerable attention has focused on NIR luminescence contrast agents and there is a continuing need for contrast agents containing a luminescent substance where the luminescent substance provides a reasonably high quantum efficiency, and where the emission resulting from the luminescent substance predominantly emits in a spectral range where there is little background, especially background resulting from autofluorescence of the tissue or blood.

Furthermore, it is known that the spectral range of excitation, i.e. the spectral range where the contrast agent and the tissue absorbs the light, may determine whether the tissue surface or deeper located tissue areas are accessible for optical imaging. Consequently, it would be desirable to provide a contrast agent containing a luminescent substance which absorbs over a broad spectral range, so that the contrast agent is suitable for imaging the tissue surface and the deeper located tissue areas.

It is an object of the present invention to provide a contrast agent containing a luminescent substance having reasonably high quantum efficiency and, at the same time, emitting in a spectral range where no or little autofluorescence is observed.

It is also an object of the present invention to provide a contrast agent containing a luminescent substance which absorbs over a broad spectral range and preferably has strong absorption bands in the UV as well as in the VIS spectral range.

In order to achieve the aforementioned object, a contrast agent as defined in independent claim 1 is provided.

According to one embodiment of the present invention, a contrast agent for optical imaging is provided, wherein the contrast agent comprises a luminescent substance and wherein the luminescent substance comprises Tm²⁺.

According to the present invention Tm²⁺-containing contrast agents possess several strong absorption bands over a relatively broad spectral range and, at the same time, emit mainly outside of the aforementioned range in the relevant temperature range. In other words, the absorption and the emission of the inventive contrast agent takes place in different spectral ranges which means that there is a spectral distance between absorption and luminescence maxima. More precisely, it has been found that Tm²⁺-containing luminescent substances at temperatures in the range from 300 K to 315 K (room temperature to body temperature) mainly or almost exclusively image or emit in a relatively small wavelength range in the near infrared spectral range (NIR). In view of the fact that autofluorescence is usually observed in the UV-VIS range, the emitting signals observed for the inventive contrast agent do not overlap with the signals resulting from blood and tissue. Accordingly, the optical imaging is not affected by unwanted background signals if the inventive contrast agents are used. Furthermore, the relatively broad absorption range observed for the inventive contrast agents allows for the excitation with UV light or visible light.

It is to be understood that when subsequently reference is made to the “inventive contrast agent” or the “inventive luminescent substance”, it is referred to any of the embodiments described herein. The term “tissue” as used herein also encompasses organs and cells.

In one embodiment of the present invention, the inventive luminescent substance absorbs electromagnetic radiation in the wavelength range between 200 nm and 800 nm and preferably in the range 350 nm and 700 nm at a temperature in the range from 300K to 315K.

In a further embodiment of the present invention, the inventive contrast agent contains a luminescent substance imaging or emitting electromagnetic radiation in the near infrared wavelength range between 750 nm and 1400 nm, preferably in the wavelength range between 950 and 1300 nm and even more preferred in the range between 1050 nm and 1150 nm at a temperature in the range of 300K to of 315K.

In yet another embodiment of the present invention, the inventive luminescent substance is formed of inorganic solids.

According to further embodiments of the present invention, the inventive contrast agent contains Tm²⁺ doped into alkaline earth metal halides such as CsCaCl₃, CsCaBr₃ and/or CsCaI₃. Alternatively, the inventive contrast agent may contain TmX₂, wherein X is selected from F, Cl, Br, I and At.

According to another preferred embodiment, the luminescent substance is in the form of nanocrystals, wherein the nanocrystals may be modified or functionalized.

In one embodiment of the present invention, the inventive contrast agent does not contain detectable amounts of Tm³⁺.

In yet another embodiment, the inventive contrast agent comprises a core and a shell, wherein the core contains the luminescent substance and wherein the shell may comprise a biocompatibility improving material or a material containing at least one antibody or a functionalized material having a specific affinity to a certain location or a certain tissue within the human body.

Furthermore, the present invention refers to the use of a Tm²⁺ containing material as a luminescent substance in a contrast agent for optical imaging. According to one preferred embodiment, the inventive contrast agent as defined in the claims or the embodiments described is used optical imaging.

According to yet another embodiment of the present invention, a method of optical imaging of tissue is provided, wherein the method comprises the following steps:
(a) contacting an effective amount of the inventive contrast agent with the tissue,
(b) exposing the tissue to electromagnetic radiation in the wavelength range between 200 nm and 800 nm at a temperature in the range from 300K to 315K,
(c) detecting any luminescence signal emitted by the tissue exposed to the electromagnetic radiation at a temperature in the range from 300K to 315K, and
(d) processing the detected luminescence signal(s) into an image.

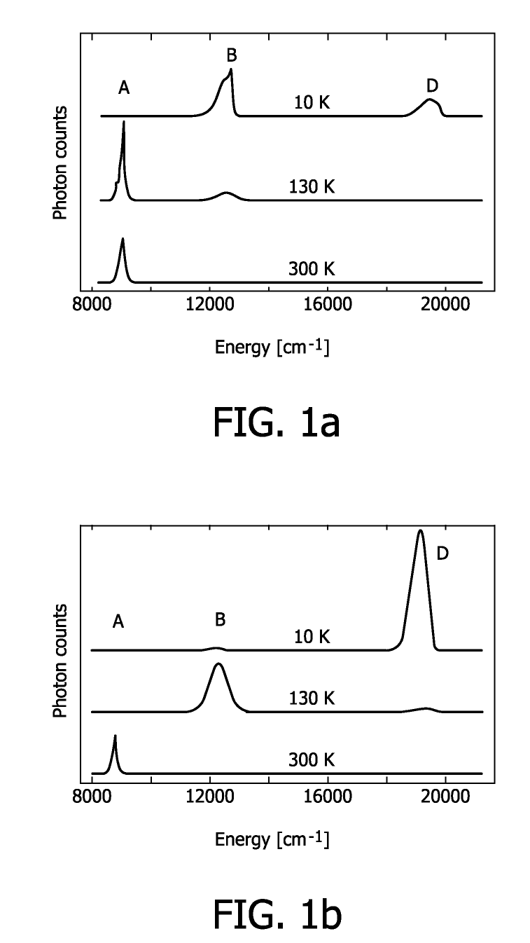

FIG. 1 shows the emission spectra of Tm²⁺ in (a) CsCaCl₃, and (b) CsCaBr₃ at 300K. The emission was photoexcited at 21834 cm⁻¹ corresponding to about 458 nm. FIG. 1 corresponds to “FIG. 3” as published in “Light-Emission and Excited-State Dynamics in Tm²⁺ Doped CsCaCl₃, CsCaBr₃ and CsCal₃” by J. Grimm, J. F. Suyver, E. Beurer, G. Carver and H. U. Güdel in J. Phys. Chem. B (2006), 110 (5), 2093-2101.

As already set out above, the term “optical imaging” encompasses a variety of different methods, wherein all optical imaging methods are based on the absorption of light and the emission of luminescence. The detection of luminescent contrast agents is, at least to some extent, comparable to nuclear imaging methods, as in both methods the contrast enhancing agents are distributed within the tissue. An important advantage of luminescence over radiodiagnostic agents is that luminescent agents can be excited continuously, as well as that the agent needs not to be radioactive.

Exogenously applied optical contrast agents principally provide the opportunity of generating in vivo or in vitro disease specific signals within tissue, thus enabling the display of physiological and molecular conditions which are characteristic for a specific disease or a certain diseased state. By using contrast agents a contrast enhancement may be achieved when the administered agent changes the absorption or luminescent properties of the diseased tissue to a different extent relative to the surrounding normal tissue. The most promising approach to achieve contrast enhancement or improved optical imaging refers to the use of luminescent substances influencing the absorption and luminescence of the tissue.

The inventor surprisingly found that Tm²⁺-containing contrast agents possess several advantageous properties which make the inventive contrast agents especially suitable for optical imaging methods. Thulium is a rare earth metal having the atomic number 69. According to the present invention Thulium is used in its divalent state, denoted as Tm²⁺.

One major advantage which is provided by the use of Tm²⁺ as a luminescent substance in contrast agents refers to the fact that Tm²⁺ has several strong absorption bands in the UV-VIS spectral range, but emits mainly outside of the aforementioned range. More precisely, it has been found that Tm²⁺-containing luminescent substances mainly or almost exclusively emit in a relatively small wavelength range in the near infrared spectral range (NIR). As a consequence, the absorption and the emission of the inventive contrast agent takes place in different spectral ranges which means that there is a spectral distance between absorption and luminescent maximum. This means that an unwanted overlap or interaction is avoided. According to one preferred embodiment, the inventive contrast agent contains a luminescent substance imaging or emitting electromagnetic radiation in the wavelength range between 750 nm and 1400 nm, preferably in the wavelength range between 900 and 1300 nm and even more preferred in the range between 1050 nm and 1150 nm at a temperature in the range from 300K to 315K.

Another advantage of the inventive contrast agents refers to the fact that the agents emit outside the UV-VIS range and, thus, avoids disturbing background signals resulting from autofluorescence. If luminescence is recorded within the UV-VIS spectral region, both autofluorescence and the administered contrast agent would contribute to the observed signal. In the NIR spectral range, however, tissue autofluorescence is negligible due to the absence of indigenous NIR fluorophors. Autofluorescence arises from the absorption of light in tissue, e.g. absorption originating from oxy- and deoxy-haemoglobin or other porphyrins. Since the emitting signals observed for the inventive contrast agent do not overlap with the signals resulting from blood and tissue, the optical imaging is not affected by unwanted background signals. In other words, when using the inventive contrast agents, the detected signal nearly exclusively reveals the distribution of the contrast agent.

It represents another advantage of the inventive contrast agents that the strong absorption bands observed for Tm²⁺ allow for the excitation over a broad spectral region ranging from the UV to the VIS spectral range. One fundamental observation for optical diagnostic procedures relates to the fact that the penetration depth of light into tissue depends on the wavelength used. Usually, absorption dominates scattering if wavelengths in the lower spectral range, e.g. the UV or blue spectral range are used for excitation. Consequently, for excitation wavelengths in this range usually a small penetration depth up to a few millimeters is achieved. This means that by using wavelengths in the UV to blue spectral range, it is possible to examine tissue surfaces with relatively high spatial resolution. It is clear that a contrast agent to be used for such optical imaging techniques must absorb light in the corresponding spectral range.

If it is desired to achieve penetration depths up to a few centimeters for imaging larger tissue volumes, electromagnetic radiation in spectral range above the blue spectral range may be preferred. By exciting the tissue with light in a spectral range of e.g. 550 to 750 nm, the identification of inhomogeneities in the bulk tissue may be achieved. Due to scattering, photons do not follow straight paths when propagating through tissue. Therefore, known mathematical models of photon transport should be used to visualize or determine the optical properties of tissue.

Since the choice of the spectral range of absorption may allow for adjusting whether a contrast agent is mainly detectable on tissue surface or deeper located tissue areas and since in many cases contrast agents have only a small range of absorption, it is often not possible to use one contrast agent for imaging tissue surfaces and deeper located tissue areas. However, the inventive contrast agent has several strong absorption bands ranging from the UV to the VIS spectral region and, thus, allow for optical imaging of tissue surface and deeper located areas.

Another prerequisite for sensitive detection of a contrast agent is a high extinction coefficient at the desired absorption wavelength. The foregoing requirement is fulfilled by the inventive contrast agents since the Tm²⁺ containing luminescent substances provides molar absorptivity coefficients which are in the range of 100-1000 M⁻¹ cm⁻¹, which is 4 to 5 orders of magnitude greater than those typically found for trivalent rare earth ions (e.g. ˜0.1 M⁻¹ cm⁻¹ for Eu³⁺).

According to the present invention, known imaging techniques can be used. In view of the fact that the Tm²⁺ containing contrast agents according to the present invention can be excited at wavelength in the UV to blue spectral range (small penetration depth) and also in the a spectral region ranging e.g. from 550 nm to 750 nm (penetration depths up to a few centimeters), the inventive contrast agents may be suitable for imaging of superficial objects where reflected or scattered photons are directly measured and may also be suitable for diffuse imaging where photons are recorded after passing through relatively thick tissue and optical properties of the tissue are spatially reconstructed using mathematical models. These applications may require different technical resolutions and instrumental geometries which, however, are known to the skilled person.

Images from superficial structures are generally obtained in reflection geometry by illumination with light of a desired wavelength and detection with suitable devices like e.g. CCD cameras or photomultipliers. For imaging larger tissue volumes illumination geometries may be used such that defined tissue areas are illuminated with light, and the transmitted scattered light is detected in a 1800 projection geometry. Stepwise scanning of the tissue area and image reconstruction from each single measurement of transmitted light intensity yields two-dimensional projection images. Three-dimensional optical images can be obtained, e.g. by diffused optical tomography (DOT) which is based on the detection of photons at multiple positions/angles and the mathematical reconstruction of corresponding images.

The excitation of the inventive contrast agents can be achieved by a variety of different light sources, including incandescent lamps, fluorescent lamps, gas discharge lamps, laser light, LEDs (light emitting diode), e.g. a GaN- or InGaN-based LED. All these types may be mounted to the end of an endoscope. If appropriate, the light of such a source may be directed through an optical fiber to the head of an endoscope or another minimally invasive device.

The inventive contrast agents may be used in optical imaging methods for visualizing ocular diseases, chorioretinal diseases, such as vascular disorders, retinopathies, neovascularization or tumors. Furthermore, human brain tumors and tumor margins during surgery may be identified with the help of the inventive contrast agents, thereby facilitating the accuracy and safety of tumor resections and minimizing the probability of tumor recurrence. The inventive contrast agents may also be used in optical imaging techniques for visualizing superficial diseases in hollow organs using luminescence guided endoscopy. Exemplarily, reference is made to urinary bladder cancer, bronchial cancer, various gastro-intestinal diseases and tumors in the oral cavity. Also the use of flexible endoscopes may be advantageous. By using flexible catheters, intravascular luminescent spectroscopy and imaging helps identify arterial sclerotic plaques and other vascular abnormalities. Diffuse optical tomography (DOT) carried out with the inventive contrast agents may provide the opportunity to quantify cerebral blood flow and oxygenation in the brain.

As explained above, the relatively broad absorption range provided by the inventive Tm²⁺-containing contrast agents allows for the provision of contrast agents which can be used for both of the aforementioned classes of optical imaging since the inventive contrast agents possess strong absorption bands in the UV to blue spectral range and have strong absorption bands in the spectral range above 550 nm. Consequently, the inventive contrast agents may be used in optical imaging techniques for imaging the outer regions of tissue, e.g. by using endoscopic techniques and for in-depth imaging, i.e. for visualizing abnormalities in the bulk tissue. In both cases, the imaging signals resulting from the inventive contrast agent do not overlap with any background signals or signals resulting from autofluorescence. It may be advantageous to use a light source imaging or exciting the contrast agent with electromagnetic radiation in the wavelength range between 200 nm and 800 nm and preferably in the range 350 nm and 700 nm in order to ensure that there is a sufficient spectral distance between the emitting signal and the spectral range of absorption.

In a preferred embodiment of the present invention the luminescent substances are formed of inorganic solids or inorganic salts. These are usually easy to handle and easy to obtain. The luminescent substances may be selected from the group comprising Tm²⁺-doped CsCaCl₃, Tm²⁺-doped CsCaBr₃ and Tm²-doped CsCaI₃, TmF₂, TmCl₂, TmBr₂, TmI₂ and TmAt₂ as well as carbonates, phosphates, hydroxides, sulphides, sulphates and chromates of Tm²⁺. Halogenides of Tm²⁺ can be formed by, e.g. reacting Tm³⁺ halogenides with hydrogen or with elemental thulium. The oxide of Tm²⁺ is obtainable e.g. by reducing Tm₂O₃ with elemental thulium.

It may be especially preferred that the inventive contrast agent contains Tm²⁺ doped CsCaCl₃, CsCaBr₃ and/or CsCaI₃.

It will be apparent to those skilled in the art that Tm²⁺ or Tm²⁺-containing substances may also be amenable to chelation. A chelator may comprise an organic, covalent, bridge-ligand molecule, capable of partly or entirely surrounding the Tm²⁺ or a Tm²⁺-containing substance. According to another embodiment of the present invention, the Tm²⁺ or Tm²⁺-containing substance is stabilized or complexed by ligands. The ligands may form a cage-like complex or structure around the Tm²⁺ or Tm²⁺-containing substance. The ligands may be polydentate ligands, preferably polydentate ligands with a selective high affinity to certain binding sites that can be used in a variety of applications in a manner analogous to the use of antibodies. Such polydentate ligands with a selective high affinity typically comprise a multiplicity of ligands that each bind different regions on the target molecule or target binding site (e.g. in a specific tissue). The ligands are joined directly or through a linker thereby forming a polydentate moiety that typically binds the target molecule with high selectivity and avidity.

According to the present invention, it is especially preferred that the inventive contrast agent contains no Tm³⁺ in detectable amounts. Tm³⁺-containing substances may provide additional emitting signals (e.g. in the autofluorescence range) and, therefore, would affect the quality of the optical imaging results. Furthermore, presence of Tm³⁺ will adversely influence the stability of the desired Tm²⁺ fraction present in the contrast agent.

According to another preferred embodiment of the present invention, the luminescent substance is in the form of nanocrystals or nanoparticles, which may be additionally functionalized. The use of a nanoparticulate form may facilitate the handling and administration of the contrast agent. According to the present invention, the nanoparticles contain Tm²⁺. According to yet another preferred embodiment, the luminescent substances are formed of nanocrystals and may be surrounded or overcoated by a shell or outer layer.

One method for preparing nanocrystals is based on the pyrolysis of organometallic precursors in hot coordinating agents. Coordinating agents can help control the growth of the nanocrystal. A coordinating agent may be a compound having a donor electron pair that, for example, is available to coordinate to a surface of the growing nanocrystal. Solvent coordination can stabilize the growing nanocrystal. Typical coordinating agents include alkyl phosphines, alkyl phosphine oxides, or alkyl phosphonic acids. Other coordinating agents such as pyridines, furans and amines may also be suitable for the nanocrystal production. The nanocrystals according to the present invention can be spheres, rods, discs or other shapes.

Inventive nanocrystals may be stabilized by monodentate or polydentate ligand on the surface of the nanocrystal. Suitable polydentate ligands may be polyphosphines, polyphosphine oxides, polyphosphinic acids, thiols or a polyphosphonic acid, or salts thereof. Advantageously, polydentate ligands, particularly oligomerized polydentate ligands such as polydentate oligomerized phosphine ligands, bind more strongly to the surface of the nanocrystal than monodentate ligands. Polydentate ligands thus stabilize the nanocrystal, which can preserve the high luminescence of as-grown nanocrystals. The polydentate ligands are chemically flexible so that they can be easily functionalized to be compatible with a specific chemical environment. The oligomeric ligands may form a passivating and/or a functionalized layer. The functionalized layer can deliver desirable chemical properties including solubility, miscibility and other derivatizations such as conjugation to biomolecules, e.g. biomolecules having a high affinity to specific types of tissues.

As a particular advantage of the present invention, the corresponding luminescent substances absorb and emit electromagnetic radiation at different wavelengths. This leads to a clear distinction between the radiation applied to the contrast agent and the radiation obtained therefrom. As a consequence, it is possible to detect radiation emitted from the contrast agent despite excitation radiation, which is used to excite the luminescence, being present, or even in the presence of background light. It is understood that this positive effect increases with the distance between the wavelength ranges in which the absorption and emission takes place as well as with the accuracy and precision of the spectral areas in which absorption and emission takes place. As a particular advantage, exciting radiation at e.g. a wavelength range of 400 to 500 nm is identified and characterized by its blue or violet appearance. Accordingly, any luminescence or light coming from the probe that is not blue or violet may be identified as some result or effect. It may be generally advantageous if the exciting radiation lies in the range beneath the visible wavelength range.

Furthermore, the luminescent substances according to the present invention preferably emit electromagnetic radiation in the wavelength range between about 750 and 1400 nm at a temperature in the range of 300K to of 315K and more preferably in the wavelength range between about 1050 and 1150 nm. As can be gathered from FIG. 1, the inventive contrast agent or luminescent substances have one strong emitting signal in the near infrared range, whereas the absorption and excitation takes places in the spectral range covering the UV area and the visible light area (VIS). Thereby it is ensured in a favorable manner that light emitted from these materials is sufficiently different form the excitation radiation so that a contrast generation is permissible. The emitted light may be detected spectroscopically.

According to another preferred embodiment of the present invention, the contrast agent has a core/shell structure, which means that the contrast agent is composed of a core comprising a Tm²⁺-containing luminescent substance and at least one shell which partly or completely surrounds said core. The luminescent core may be in the form of nanocrystals or nanoparticles.

The luminescent core may be provided with an additional layer or surrounded by a shell in order to achieve a high biocompatibility. In other words, a shell material may be used in order to prevent an immune reaction of the examined body against the contrast agent particles. Alternatively, a shell may be provided for achieving a favorable or targeted distribution of the contrast agent in the tissue to be examined. In this context, biological active compounds, such as antibodies may be used as shell materials. Furthermore, by using a shell around the luminescent core, the solvation or hydrolysis of the core or the luminescent substance may be prevented (if the luminescent core is formed of a material that is sensitive to hydrolysis). Finally, by using a shell around the luminescent core, the luminescence quantum efficiency can be substantially increased.

According to one preferred embodiment of the present invention, the shell material provides an improved biocompatibility (in comparison to a contrast agent comprising the luminescent core without a layer or shell). By using at least one biocompatibility improving shell material, the contrast agent may not cause any immune reaction against the agent after administering the contrast agent to a living organism or at least may reduce the risk of an immune reaction. Furthermore, toxic interactions may be prevented. It may be especially preferred if the at least one biocompatibility improving material covers the luminescent core completely, in order to efficiently provide biocompatibility and in order to prevent the core from being dissolved or hydrolyzed.

The at least one biocompatibility improving material according to the present invention may be selected from a polyphosphate, an amino acid, an organic polymer like polyethylene glycol (PEG) or polyvinyl alcohol (PVA), a biopolymer like polysaccharide (e.g. dextrane, cellulose), a polypeptide, a phospholipid, SiO₂ or gold. By using gold as shell material, some further positive effects may be achieved. For example, further active or inactive compounds like certain polypeptides, proteins or antibodies may be immobilized or bound on the gold surface (e.g. via thiole linkers).

According to another preferred embodiment of the present invention, the inventive contrast agent comprises at least one shell material containing at least one antibody. By immobilizing antibodies on the surface of the shell or the luminescent core, specific antibody-antigen reaction and, thus, specific adsorption of the contrast agent in the tissue to be examined (e.g. cancer cells, coronary plaques) may be achieved. This means that higher concentrations of the contrast agent in a specific type of tissue can be provided. The enriched contrast agent in the tissue may lead to better optical imaging results.

According to another preferred embodiment of the present invention, the inventive contrast agent comprises at least one shell material containing at least one antibody, wherein the at least one antibody containing shell may contain a tumor specific antibody. The use of a tumor specific antibody may allow for the use of the inventive contrast agents for the identification and localization of specific tumors. Exemplary, reference is made to Cetuximab (detection of bowel cancer), Pemtumomab (detection of ovary and stomach cancer) and Bevacizumab (detection of lung and bowel cancer). The corresponding inventive contrast agents may be especially suitable for optical imaging and diagnoses of malign changes of throat, gullet, stomach or intestine.

According to another preferred embodiment of the present invention, the size of the core or the contrast agent comprising a core and a shell may be in the range of the size of proteins and bioorganic compounds as present in human and animal organisms in order to get them more easily involved in metabolism processes, as for example intercellular exchange reactions, thereby facilitating the transport and adsorption of the contrast agents at areas of interest. The size of the core or the contrast agent comprising a core and a shell may be in the range of 50 nm to 1000 nm, preferably 100 nm to 500 nm.

The contrast agents, being in the form of nanocrystals or any other form according to the present invention can be incorporated into formulations, such as an injectable preparation that can include any acceptable diluent, or a slow-release matrix in which the contrast agent is embedded. The formulation can be provided in a container, pack or dispenser together with instructions for administration. The composition can be formulated in accordance with the intended route of administration. Acceptable routes include oral or parenteral routes, e.g. intravenous, transdermal or transmucosal. The formulation can be formulated as a solution or suspension and, thus, can include a sterile diluent (e.g. water, saline solution, a fixed oil, polyethylene glycol etc.) Where necessary, the pH of the solution or suspension can be adjusted with an acid or a base. Proper fluidity can be maintained by a coating such as lecithin by maintaining a required particle size or by the use of surfactants.

According to a further aspect of the present invention, a pharmaceutical formulation is provided which comprises the inventive contrast agent according to any of the above described embodiments and a pharmaceutically acceptable carrier or excipient, wherein the carrier may contain a physiologically acceptable compound that acts, e.g. to stabilize the formulation or the contrast agent or may regulate (increase or decrease) the absorption of the agent and/or pharmaceutical formulation. Suitable physiologically acceptable compounds may be selected from carbohydrates, such as glucose, sucrose, or dextrans, antioxidants, such as ascorbic acid or glutathione, low molecular weight proteins or other stabilizers and/or buffers. Furthermore, detergents may be used to stabilize the formulation or may (increase or decrease) the absorption of the pharmaceutical formulation. Other physiologically acceptable compounds include wetting agents, emulsifying agents, dispersing agents or preservatives that are particularly useful for preventing the growth or action of microorganisms.

The inventive formulation may contain pharmaceutically acceptable auxiliary substances for providing physiological conditions such as pH adjusting and buffering agents, toxicity adjusting agents and the like. Exemplary, reference is made to sodium acetate, sodium chloride, potassium chloride and calcium chloride.

The concentration of contrast agent in the inventive formulations can vary widely, and will be selected primarily based on parameters like fluid volumes, viscosities, body weight and the like and in accordance with the particular mode of administration and imaging modality selected. The exact amount and concentration of contrast agent of the invention and the amount of formulation in a given dose, or the “effective dose” can be routinely determined. Due to the very high sensitivity for near infrared light (e.g. through the use of single-photon counters) as well as due to the high specificity (due to the absence of tissue autofluorescence in this wavelength regime), low concentrations can oftentimes suffice. Typical values can be in the micromolar range, even down to the nanomolar range when the area/volume under investigation is limited.

The contrast agent can be formulated in accordance with the intended route of administration. Acceptable routes include oral or parenteral routes, e.g. the intravenous, transdermal or transmucosal route. The formulation can be formulated as a solution or suspension and, thus, can include a sterile diluent (e.g. water, saline solution, a fixed oil, polyethylene glycol etc.)

According to another embodiment of the present invention, a method of optical imaging of tissue is provided, wherein the method comprises the following steps:
(a) contacting an effective amount of the inventive contrast agent according to any of the afore mentioned embodiments with the tissue,
(b) exposing the tissue to electromagnetic radiation in the wavelength range between 200 nm and 800 nm at a temperature in the range of 300K to of 315K;
(c) detecting any luminescence signal in the near infrared spectral range emitted by the tissue exposed to the electromagnetic radiation at a temperature in the range of 300K to of 315K, and
(d) processing the detected luminescence signal(s) into an image.

According to another aspect of the present invention a method for in vivo or in vitro imaging and/or diagnosis of a cell, a tissue, an organ or a full body is provided, wherein the method comprises the following steps:
a) providing a pharmaceutical formulation comprising the inventive contrast agent according to any of the above described embodiments and a pharmaceutically acceptable excipient or carrier,
b) providing an optical imaging device;
c) administering the pharmaceutical formulation in an amount sufficient to generate the cell, tissue or body image; and
d) imaging the distribution of the pharmaceutical formulation of step a) with the imaging device, thereby imaging the cell, tissue or body.

In one embodiment of the present invention, in the aforementioned methods a Tm²⁺-containing luminescent substances may be used that absorbs electromagnetic radiation in the wavelength range between 200 nm and 800 nm and preferably in the range 350 nm and 700 nm at a temperature in the range of 300K to of 315K.

In a further embodiment, in the aforementioned methods a Tm²⁺-containing luminescent substances may be used that emits electromagnetic radiation in the wavelength range between 750 nm and 1400 nm, preferably in the wavelength range between 950 and 1300 nm and even more preferred in the range between 1050 nm and 1150 nm at a temperature in the range of 300K to of 315K.

It may be especially preferred that the inventive contrast agent to be used in the aforementioned methods contains Tm²⁺ doped CsCaCl₃, CsCaBr₃ and/or CsCaI₃.

It may be also especially preferred that the inventive contrast agent to be used in the aforementioned methods comprises a core containing the luminescent substance and a shell, wherein the shell may comprise a biocompatibility improving material or a material containing at least one antibody. Reference is made to the antibody containing materials mentioned above.

The inventive methods of diagnosing and/or imaging may be used to diagnose and/or detect a cancer selected from the group comprising leukemia, lymphoma, brain cancer, cerebrospinal cancer, bladder cancer, prostate cancer, breast cancer, cervical cancer, uterus cancer, ovarian cancer, kidney cancer, oral and throat cancer, esophagal cancer, lung cancer, colonorectal cancer, pancreatic cancer, and melanoma.

While the present invention has been described with respect to specific embodiments thereof, it will be recognized by those of ordinary skill in the art that many modifications, enhancements, and/or changes can be achieved without departing from the spirit and scope of the invention. Therefore, it is manifestly intended that the invention be limited only by the scope of the claims and equivalents thereof. In the following, the invention is illustrated in view of certain examples. These examples are however in no way meant to limit the invention as to its scope, but rather serve to illustrate the invention by way of some of its exemplary embodiments.

### EXAMPLES

Single crystals of CsCaCl₃, CsCaBr₃ and CsCaI₃ doped with Tm²⁺ were grown by the Bridgeman Technique. For the synthesis, stoichiometric amounts of CsX (X═Cl, Br, I) and CrX₂ were mixed and Tm²⁺ was prepared in situ by synproportionation of TmX₃ (prepared with the ammonium halide route from 99.999% pure Tm₂O₃ from Johnson Matthey) and Tm metal (Alpha Aesar 99.9%). All starting materials are hygroscopic and were handled in a glove box on a nitrogen atmosphere. Dark green crystals of good optical quality with diameters up to 2 mm×2 mm×2 mm were obtained. The crystals were checked for purity by X-ray powdered diffraction. The absolute concentrations of Tm in the crystals were determined with ICP-OAS and are 1.04%, 0.48%, and 0.76% CsCaCl₃, CsCaBr₃ and CsCaI₃, respectively. Tantalum ampules were used for obtaining crystals. The obtained crystals may be processed into a suitable formulation which may be used as contrast agent for optical imaging.

1. A contrast agent for optical imaging comprising a luminescent substance, wherein the luminescent substance comprises Tm2+.

2. Contrast agent according to claim 1,
wherein the luminescent substance absorbs electromagnetic radiation in the wavelength range between 200 nm and 800 nm at a temperature in the range from 300K to 315K.

3. Contrast agent according to claim 1,
wherein the luminescent substance absorbs electromagnetic radiation in the wavelength range between 350 nm and 700 nm at a temperature at a temperature in the range from 300K to 315K.

4. Contrast agent according to claim 1,
wherein the luminescent substance emits electromagnetic radiation in the wavelength range between 750 nm and 1400 nm at a temperature at a temperature in the range from 300K to 315K.

5. Contrast agent according to claim 1, wherein the luminescent substance emits electromagnetic radiation in the wavelength range between 950 nm and 1300 nm at a temperature at a temperature in the range from 300K to 315K.

6. Contrast agent according to claim 1,
wherein the luminescent substance emits electromagnetic radiation in the wavelength range between 1050 nm and 1150 nm at a temperature in the range from 300K to 315K.

7. Contrast agent according to claim 1,
wherein the luminescent substance is formed of inorganic solids.

8. Contrast agent according to claim 1,
wherein the contrast agent contains at least one of Tm2+ doped CsCaCl3, Tm2+ doped CsCaBr3 and Tm2+ doped CsCaI3.

9. Contrast agent according to claim 1,
wherein the contrast agent contains TmX2, wherein X is selected from F, Cl, Br, I and At.

10. Contrast agent according to claim 1,
wherein the luminescent substance is in the form of nanocrystals.

11. Contrast agent according to claim 1,
wherein the luminescent substance is in the form of functionalized nanocrystals.

12. Contrast agent according to claim 1,
wherein the luminescent substance contains no Tm3+ in detectable amounts.

13. Contrast agent according to claim 1,
wherein the contrast agent comprises a core containing the luminescent substance and a shell.

14. Contrast agent according to claim 13,
wherein the shell comprises a biocompatibility improving material or a material containing at least one antibody.

15. Use of a contrast agent according to claim 1 for optical imaging.

16. A method of optical imaging of tissue using a contrast agent according to claim 1, the method comprising the steps of:
(a) contacting an effective amount of the contrast agent with the tissue,
(b) exposing the tissue to electromagnetic radiation in the wavelength range between 200 nm and 800 nm at a temperature in the range from 300K to 315K;
(c) detecting any luminescence signal emitted by the tissue exposed to the electromagnetic radiation, and
(d) processing the detected luminescence signal(s) into an image.

17. A method of imaging and/or diagnosis of a cell, a tissue, an organ or a full body, wherein the method comprises the following steps:
a) providing a pharmaceutical formulation comprising a contrast agent according to claim 1 and a pharmaceutically acceptable excipient or carrier,
b) providing an optical imaging device;
c) administering the pharmaceutical formulation in an amount sufficient to generate the cell, tissue or body image; and
d) imaging the distribution of the pharmaceutical formulation of step a) with the imaging device, thereby imaging the cell, tissue or body.
